(19) United States
(12) Patent Application Publication
(10) Pub. No.: US 2026/0047857 A1
(43) Pub. Date: Feb. 19, 2026

(54) Surgical Assembly and System

(71) Applicant: Alesi Surgical Limited, Cardiff (GB)

(72) Inventors: Jason BREWER, Sigingstone, Cowbridge (GB); Richard CURTIS, Crosskeys, Newport (GB); Francis AMOAH, Shinfield, Reading (GB)

(73) Assignee: Alesi Surgical Limited, Cardiff (GB)

(21) Appl. No.: 18/868,726

(22) PCT Filed: Apr. 27, 2023
(86) PCT No.: PCT/GB2023/051116

§ 371 Date: Nov. 22, 2024

(30) Foreign Application Priority Data
May 25, 2022 (GB) 2207678.0

(51) Int. Cl. A61B 17/32; A61B 17/00

(52) U.S. Cl. CPC A61B 17/320068; A61B 2017/00106

(57) ABSTRACT

A surgical assembly is disclosed for use with an ultrasonic surgical device that is arranged to deliver ultrasonic vibrations to patient tissue for use in cutting or cauterizing patient tissue during a surgical procedure. The assembly comprises an ion-generating electrode arranged to receive an electrical signal for generating ions proximate a site of the surgical procedure for removing particles suspended proximate the surgical site, and a controller for controlling the application of the electrical signal to the ion-generating electrode. The assembly further comprises a sensing arrangement for sensing a presence of ultrasonic vibrations, and which is communicatively coupled with the controller and arranged to output an activation signal to the controller to cause the electrical signal to be applied to the ion-generating electrode when the sensed ultrasonic vibrations comprise a frequency within a pre-defined frequency range.

The present invention relates to a surgical assembly and system.

Many laparoscopic procedures involve the use of surgical energy devices such as lasers and diathermy devices, which can cut and cauterise tissues by ablation, heating, freezing and the like. In certain situations, particularly when the procedure is performed within a laparoscopic cavity, the surgeon's view can be become obscured by smoke and vapours, namely aerosols, which are generated during surgery, and it is therefore necessary to provide a means for extracting the aerosols from the surgical site. The extraction of aerosols can be achieved in a number of ways, such as by over-pressurising the cavity with carbon dioxide and providing a gas bleed tube fitted with a filter to circulate the gas through the surgical site. Alternatively, the aerosol may be extracted via electrostatic precipitation, such as in the manner described in WO2011/010148. Electrostatic precipitation comprises the use of an electrode for generating ions within the aerosol, so that the electrostatically charged aerosol can subsequently be captured via electrostatic attraction to a collection site. The aerosol is thus removed from the surgical site to clear the surgeons view, and transferred to the collection site where it can thereafter be removed.

When ion-generating electrodes are activated precautions must be taken to ensure that an electrical contact is not made between the electrode and other surgical instruments within the laparoscopic cavity for protracted time intervals, or with patient tissue of vulnerable organs such as the heart, since these events have the potential to create electrical shocks of different levels of severity to the patient and to the surgeon. In addition, any electrical devices connected to patients must satisfy rigorous safety standards to ensure that any electrical interference between neighbouring devices is minimised. Unlike surgical energy devices that couple low voltage DC or a reversing polarity AC waveform to a patient, surgical instruments or devices that couple high voltage DC waveforms to the patient are capable of transferring or storing electrical charge either directly or indirectly, such as by storing electrical charge within the instrument or coupling charge to an instrument of another medical device positioned within the patient body. This build-up of electrical charge can lead to an unexpected and undesirable electrical discharge.

WO2011/010148 discloses the use of an ion-generating electrode which is “always-on” and as such any patient contact with the electrode may result in an undesirable low current electrical discharge. WO2018/234803 discloses an improved arrangement where the activation of the ion-generating electrode is synchronised with the activation of the surgical device to reduce the likelihood of accidental contact with the electrode while it is active. This synchronisation can be achieved by monitoring for radio frequency (RF) disturbance such as the leakage current from transmission lines coupling an RF generator with the surgical device, for example. However, aerosols are also produced by surgical devices which utilise ultrasonic energy to perform cutting and cauterising of patient tissue, and there is currently no mechanism for externally monitoring the activation of the ultrasonic surgical device to synchronise the activation of the ion-generating electrode.

We have now devised a surgical assembly and system which addresses this problem.

According to a first aspect of the present invention, there is provided a surgical assembly for use with an ultrasonic surgical device, said ultrasonic surgical device being arranged to deliver ultrasonic vibrations to patient tissue for use in cutting or cauterizing patient tissue during a surgical procedure, the surgical assembly comprising:
an ion-generating electrode arranged to receive an electrical signal for generating ions proximate a site of the surgical procedure for removing particles suspended proximate the surgical site;
a controller for controlling the application of the electrical signal to the ion-generating electrode; and
a sensing arrangement for sensing a presence of ultrasonic vibrations;
wherein the sensing arrangement is communicatively coupled with the controller and arranged to output an activation signal to the controller to cause the electrical signal to be applied to the ion-generating electrode when the sensed ultrasonic vibrations comprise a frequency within a pre-defined frequency range the assembly further comprising a frequency mixer for heterodyning the ultrasonic vibrations sensed by the sensing arrangement with a characteristic frequency.

In an embodiment, the pre-defined frequency range comprises a frequency associated with the ultrasonic vibrations delivered by the ultrasonic surgical device.

In an embodiment, the sensing arrangement comprises a transducer, such as a microphone, for capturing ultrasonic vibrations, and a passband filter for removing ultrasonic vibrations which comprise a frequency outside the pre-defined range.

The sensing arrangement may further comprise a threshold detector for detecting the ultrasonic vibrations admitted by the passband filter, within the pre-defined frequency range, which comprise an amplitude above a threshold level. The threshold detector is further arranged to detect the ultrasonic vibrations within the pre-defined frequency range which exist for a temporal duration greater than a threshold duration.

In an embodiment, the sensing arrangement is arranged to output the activation signal when the ultrasonic vibrations comprise a frequency within the pre-defined frequency range and when the ultrasonic vibrations comprise an amplitude above the threshold level.

In an alternative embodiment, the sensing arrangement is arranged to output the activation signal when the ultrasonic vibrations comprise a frequency within the pre-defined frequency range and when the ultrasonic vibrations comprise an amplitude above the threshold level and when the ultrasonic vibrations exist for a temporal duration greater than the threshold duration.

The predefined frequency range comprises the range between 20 kHz and 70 kHz, and preferably the range between 30 kHz and 60 KHz.

In an embodiment, the characteristic frequency comprises a frequency of ultrasonic vibrations generated by a known ultrasonic surgical device, so that the ultrasonic surgical device can be identified, and comprises at least one frequency from the group comprising 36 kHz, 47 kHz and 55 kHz. However, the assembly may be configured to be unresponsive to continually present ultrasonic vibrations, even at frequencies characteristic of known ultrasonic surgical devices (as these vibrations are not consistent with the duty cycle of the use of surgical tools and may for instance be representative of a nuisance signal source).

In an embodiment, the assembly is configured to only be responsive to a single frequency or to be unresponsive to one or more known device operating frequencies.

According to a second aspect of the present invention, there is provided a surgical system comprising a surgical assembly according to the first aspect, an electrical generator electrically coupled with the ion-generating electrode for delivering the electrical signal to the ion-generating electrode, and an ultrasonic energy generator communicatively coupled with an ultrasonic surgical device for delivering ultrasonic vibrational energy to the ultrasonic surgical device.

Embodiments of the invention will now be described by way of example only and with reference to the accompanying drawings in which:

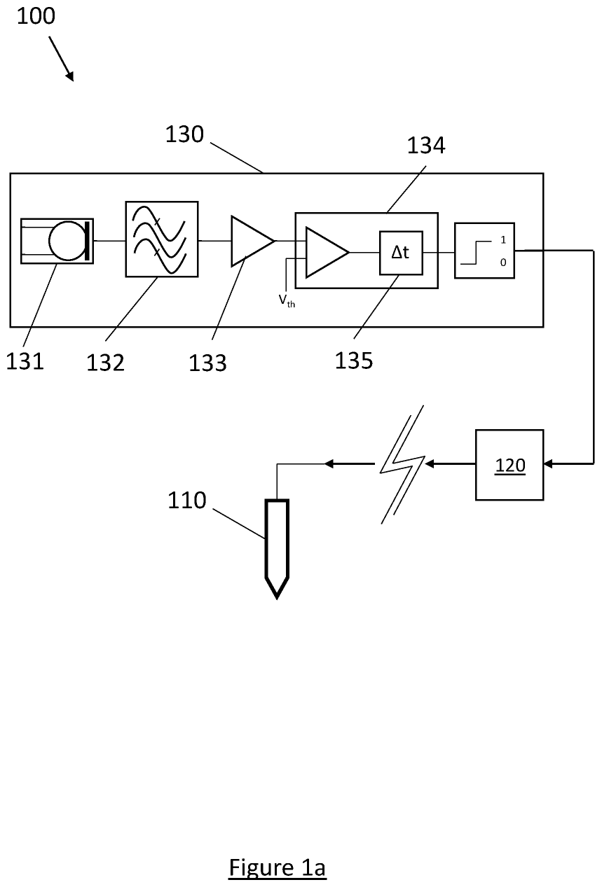

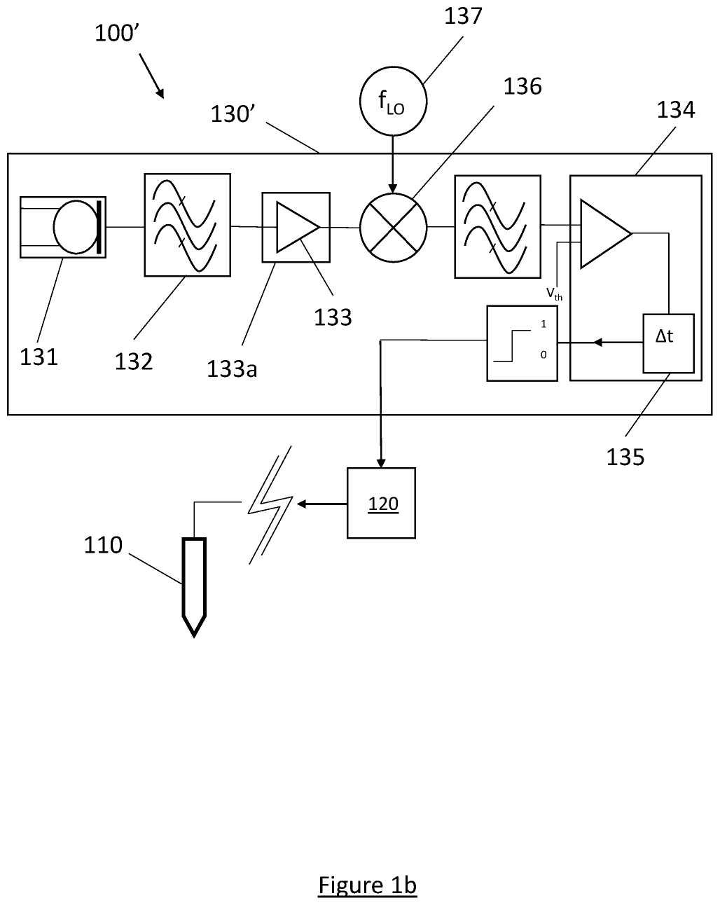

FIG. 1a is a schematic illustration of a surgical assembly according to an embodiment of the present invention;

FIG. 1b is a schematic illustration of a surgical assembly according to an alternative embodiment of the present invention; and,

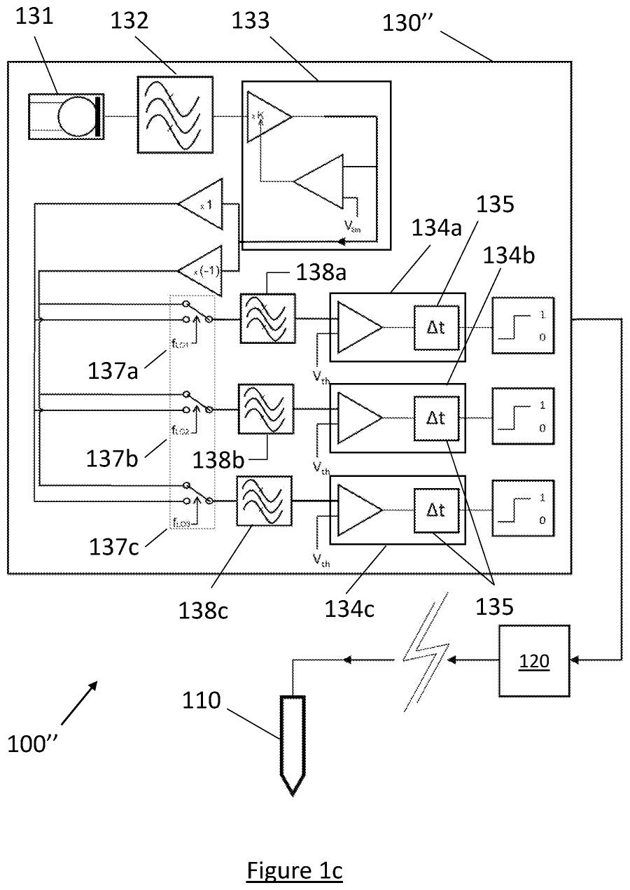

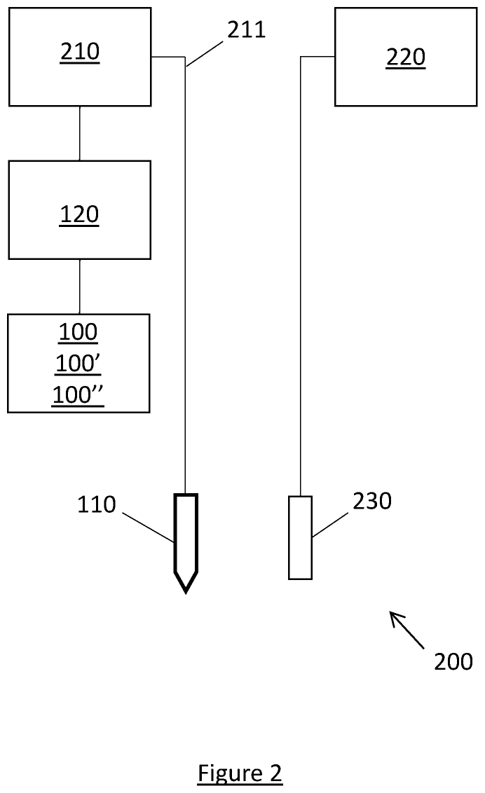

FIG. 2 is a schematic illustration of a surgical system according to an embodiment of the present invention.

Referring to FIG. 1a of the drawings, there is shown a schematic illustration of a surgical assembly 100 according to a first embodiment of the present invention. The assembly 100 is arranged to detect the activation of an ultrasonic surgical device 230 (see FIG. 2) so that an electrostatic precipitation of surgical aerosols (not shown), such as surgical smoke, can be synchronised with the operation of the surgical device 230 to maintain a clear view of the surgical site for the surgeon.

The assembly 100 comprises an ion-generating electrode 110 typically in the form of an exposed portion of wire, or a sharpened rod, for example. The electrode 110 is arranged to ionise surgical aerosol so that the aerosol can be removed from the surgical site via electrostatic precipitation. The electrode 110 is coupleable with a pole of a high voltage DC electrical generator 210 (see FIG. 2) using transmission cables 211 and is arranged to emit a stream of electrons from the exposed end of the electrode 110. These electrons attach themselves to the particles within the aerosol, thereby electrostatically charging the aerosol, which can then be subsequently attracted to a collection site (not shown), such as an electrode pad, gauze or similar, or the patient body, which is electrically coupled with the opposite pole of the electrical generator 210.

The assembly 100 further comprises a controller 120 for controlling the application of the DC electrical signal to the ion-generating electrode 110 for selective activation of the electrode 110 and thus the charging of the surgical aerosol. The controller 120 is arranged to output an activation signal to the electrical generator 210 to selectively admit the electrical signal to the ion-generating electrode in dependence of a signal output from a sensing arrangement 130.

Referring to FIG. 1a of the drawings, the sensing arrangement 130 comprises a transducer 131, such as a microphone, comprising a frequency response which extends into the ultrasonic frequency band, such as a Knowles ultrasonic microphone (manufacturer no. SPU410LR5H-QB). The transducer 131 is communicatively coupled with a passband filter 132 and is arranged to output a signal to the filter. The filter 132 is arranged to process the signal output from the transducer 131 and remove ultrasonic frequency components outside of a pre-defined range, namely outside of the passband. The sensing arrangement 130 further comprises an amplifier 133 which is arranged to receive a signal output from the filter 132 and amplify the signal. The amplified signal is then passed to a threshold detector 134 where the amplified signal is compared with a threshold level. It is envisaged that such detection thresholds may be adjustable or adaptive within a limited range to accommodate differences in ultrasonic surgical device sound signal strength at the transducer 131, against background interference from non-surgical instrument noise sources. However, it is also envisaged that the passband may comprise an adjustable centre frequency to enable discriminative responsiveness to interference from non-instrument noise sources (not shown), and to maximise the detector amplified signal strength arising from operation of the ultrasonically operating surgical instrument.

In an alternative embodiment, the threshold detector 134 is further arranged to detect the temporal duration of the amplified signal via a timer 135 and compare the duration with a threshold duration, such as 500 ms. The temporal discrimination of signals detected in the frequency band facilitates the rejection of impulse noise sources from impacts between hard surfaces which might be expected to generate a short term peak in the ultrasonic spectrum.

Known ultrasonic surgical devices operate at distinct ultrasonic frequencies. For example, the BOWA Lotus device operates at 35.8-36.6 kHz, the Olympus Thunderbeat device operates at 47 kHz, the Medtronic Sonicsion Cordless device operates at 56 kHz+1 kHz and the Ethicon Harmonic Scalpel device operates at 55.5 kHz+1 kHz. The predefined frequency range, namely the passband of the filter 132, therefore comprises a range of 20 kHz-70 kHz, and more preferably 30 kHz-60 kHz, and so extends across the typical operating frequencies of the known ultrasonic surgical devices. Having a restricted passband minimises possible signal saturation by frequencies out of the range of interest and eases bandwidth specification requirements on the signal amplifiers, so in environments where there is little or no interference or noise from other sources of ultrasonic energy, this assembly 100 can be used to detect the activation of a known ultrasonic surgical device.

Referring to FIG. 1b of the drawings, there is illustrated a surgical assembly 100′according to a second embodiment of the present invention. The surgical assembly 100′illustrated in FIG. 1b is useful where it is desirable to identify the type of ultrasonic surgical device being used during the surgical procedure. The assembly 100′illustrated in FIG. 1b comprises the same principal components as those illustrated in FIG. 1a (and so the common components have been referenced with the same numeral), however, the sensing arrangement 130′further comprises an automated gain control 133a to extend the dynamic range of signal processing from the ultrasonic sensor to 500:1, and a frequency mixer 136 for heterodyning the signal output from the passband filter 132 and amplifier 133, with a signal output from a local oscillator 137. The signal from the local oscillator 137 comprises a frequency characteristic of the ultrasonic frequency utilised by a known ultrasonic surgical device. Specifically, the local oscillator frequency is centred upon or adjacent to a narrow frequency band characteristic of the ultrasonic frequencies utilised by a known ultrasonic surgical device. The detection of frequency components at or immediately adjacent to the local oscillator frequency is difficult as it is hypothetically possible to achieve a null where both signals are identical, or similarly challengingly where it is required to process very low beat frequencies. The local oscillator frequency is preferably positioned just above the expected highest frequency from the surgical instrument on the presumption that mechanical loading of the surgical instrument during use is likely to dampen instrument resonance, and so slightly drop the signature frequency for that instrument.

In this embodiment, it is envisaged that the local oscillator 137 may sweep across several characteristic frequencies so as to sample the signal from the passband filter 132 for possible frequency signatures characteristic of the frequencies used by known ultrasonic surgical devices. The frequency components at the mixer output include significant amplitudes at the absolute difference between the local oscillator singular frequency and the narrow frequency spectrum characteristic of the known ultrasonic surgical device. This signal output from the frequency mixer 137 is then passed to a narrow-band filter 138 (approximately 1-2 kHz bandwidth) centred upon or adjacent to the characteristic frequency of a known ultrasonic surgical device, for passing signals which have a frequency within the narrow-band. This is described as a down-mixed signal. As the local oscillator frequency can be set by digitally dividing a precision high frequency source (not shown), such as a crystal oscillator, there is little error arising from the signal within the passband reaching the detector input. For instance a filter bandwidth variation of 1 to 2 kHz for a 500 Hz bandwidth spectrum at 36 KHz operating frequency will result in almost no variation in a detector signal input. By comparison, an implementation of a narrow bandpass filter at precisely 36 kHz with a 1-2 kHz bandwidth would likely result in a signal amplitude vulnerable to commercially available capacitor tolerances.

In a third embodiment of the surgical assembly 100″ (where common components have again been referenced with the same numerals), as illustrated in FIG. 1c of the drawings, the ultrasound signal output from the amplifier 133 and its voltage inverted equivalent is split to several channels, with a mixer 137 implemented as 2-input multiplexers 137a-c each toggling at or adjacent to a known respective characteristic frequency. This mixing method is known to achieve a high down-mixed high signal amplitude of the order of 60 percent of the original ultrasonic signal amplitude.

The signal output from the several mixers 137a-c and their respective narrow band low pass filters 138a-c each comprise a narrow-band spectrum centred on a respective characteristic frequency. The signal output from each narrow-band filter 138a-c is then input to a respective threshold detector 134a-c for comparing the amplitude of the signal within the channel to a threshold level, and in an embodiment, also for monitoring the temporal duration of the signal via a respective timer 135. By monitoring the signal output from a particular detector 134a-c, it is possible to determine the type of ultrasonic surgical device being activated by virtue of the respective characteristic frequency.

In an alternative embodiment to the assembly depicted in FIG. 1b, out-of-band detection (namely a detection of the down-mixed signal level when the local oscillator 137 is swept across ultrasonic frequencies away from the known respective characteristic frequencies for ultrasonic surgical device) could be arranged to determine a background noise level and microphone sensitivity and accordingly adjust the amplifier gain 133′and or the detector in-band threshold (namely the detector 134a-c which detects the signal from a known ultrasonic surgical instrument), adaptively. Moreover, in situations where only one ultrasonic surgical device is being used at any given time, then the detection should only occur in one frequency band: the threshold levels could be rapidly increased at any time where several signals above threshold level are detected in a given frequency band, and then slowly decreased towards a minimum detection threshold until there is only one signal above the detected threshold. This method does not require the use of out-of-band filters to determine background noise levels.

In a further embodiment, the controller 120 could be set to ignore or increase detection thresholds for in-band signals where there was temporal persistence beyond 60 seconds and preferably beyond 30 seconds. Such signal persistence could be indicative of sound emissions coincidentally in-band but not from ultrasonic surgical instruments such as could be generated from an instrument cleaning bath with ultrasonic agitation, from fluorescent or other switch mode power supplies. As the local oscillator frequency could be progressively swept across the characteristic frequency band for a known ultrasonic surgical instrument, and 1-2 kHz beyond upper and lower extents of the expected band, the detector thresholds could be adaptively set or mapped for discrete local oscillator frequencies according to persistent background levels, with detection being determined when an increase in above the background mapped levels was detected at one of the discrete local oscillator frequencies settings within the narrow band filter bandwidth of the known ultrasonic surgical instrument frequency spectrum.

Referring to FIG. 2 of the drawings there is illustrated a surgical system 200 according to an embodiment of the present invention. The surgical system 200 may comprise the surgical assembly of the first, second or third embodiment (100, 100′, 100″) disclosed above. In addition, the surgical system 200 comprises a high voltage DC electrical generator 210 which is communicatively coupled with the ion-generating electrode 110, and an ultrasonic energy generator 220 and an ultrasonic surgical device 230, the ultrasonic energy generator 220 being configured to deliver an ultrasonic signal to the ultrasonic surgical device 230 for delivering ultrasonic vibrational energy to patient tissue. During use, the transducer 131 of the sensing arrangement 130, 130′, 130″ may be disposed remote from the surgical site, such as within a housing of the DC electrical generator 210. When the surgeon activates the surgical device 230, such as via a button (not shown) on the device or a foot pedal (not shown) for example, the sensing arrangement 130, 130′, 130″ is arranged to detect the activation by capturing ultrasonic vibrations at the transducer 131 and process the signal using the filters 132, amplifier 133 and detectors 134 of the sensing arrangement 130, 130′130″.

In situations where the sensing arrangement 130 comprises the assembly of the first embodiment, when the amplified signal is greater than the threshold level, then the detector 134 is arranged to output a logic “1”, whereas if the amplified signal is less than the threshold level, the detector 130 is arranged to output a logic “0”. In an embodiment where the detector 134 further analyses the temporal duration of the signal using a timer 135, then where the signal extends for a duration which exceeds the threshold duration, and the amplitude of the amplified signal is greater than the threshold level, then the detector 134 is arranged to output a logic “1”. Conversely, if the amplitude of the signal is less than the threshold level, or comprises a temporal duration less than the threshold duration, then the detector is arranged to output a logic “0”.

The signal output from the detector(s) constitutes an activation signal which is communicated to the controller 120. Where the activation signal comprises a logic “1”, thereby signifying the activation of an ultrasonic surgical device 230 then the controller 120 is arranged to cause an electrical signal to be applied to the ion-generating electrode 110 and thus commence electrostatic precipitation of the surgical aerosol. When the activation signal changes to “0”, thereby signifying a deactivation or no activation of the surgical device, the controller 120 is arranged to disconnect the electrical supply to the ion-generating electrode 110 and thus terminate electrostatic precipitation. In this respect, it is evident that the assembly 100 provides for a synchronisation of electrostatic precipitation with the operation of the ultrasonic surgical device 230, such that the ion-generating electrode 110 is only activated when the surgical device is being used.

Similarly, in situations where the sensing arrangement 130″ comprises the assembly of the third embodiment for example, then when the amplified signal is greater than the threshold level in a particular detector 134a-c, then that specific detector which is tuned to a particular ultrasonic surgical device, is arranged to output a logic “1”, thereby signifying the activation of a specific ultrasonic surgical device 230. Conversely, if the amplified signal is less than the threshold level in a particular detector 134a-c, then that specific detector is arranged to output a logic “0” indicating no activation, (or a deactivation). In an embodiment where the detector 134a-c further analyses the temporal duration of the signal, then where the signal extends for a duration which exceeds the threshold duration, and the amplitude of the amplified signal output from a particular detector is greater than the threshold level, then the detector 134a-c is arranged to output a logic “1”. Conversely, if the amplitude of the signal is less than the threshold level or comprises a temporal duration less than the threshold duration, then the particular detector 134a-c is arranged to output a logic “0”.

In an embodiment, the controller response to a logic “1” detector output signal is to enable the DC voltage for ionising the aerosol generated by the operation of the ultrasonically powered surgical device, and upon cessation of operation of said ultrasonically powered surgical device, the controller response to the logic “0” detector output signal is to initialise a DC voltage count down timer, where the initial count may be in the range 3 to 15 seconds and is preferably in the range 5 to 10 seconds, and to count down said timer (not shown), disabling the DC output when the timer reaches the count of zero. The count down timer initialisation value is empirically determined to correspond to the amount of time typically required to substantially clear the aerosol remaining at the cessation of application of ultrasound power to the surgical device.

From the foregoing therefor it is evident that the above described surgical assembly and system provide an effective means of detecting the activation of an ultrasonic surgical device for synchronising the electrostatic precipitation of surgical aerosol therewith.

1. A surgical assembly for use with an ultrasonic surgical device that is arranged to deliver ultrasonic vibrations to patient tissue for use in cutting or cauterizing patient tissue during a surgical procedure, the assembly comprising:
an ion-generating electrode arranged to receive an electrical signal for generating ions proximate a site of the surgical procedure for removing particles suspended proximate the surgical site;
a controller for controlling the application of the electrical signal to the ion-generating electrode; and
a sensing arrangement for sensing a presence of ultrasonic vibrations;
wherein the sensing arrangement is communicatively coupled with the controller and arranged to output an activation signal to the controller to cause the electrical signal to be applied to the ion-generating electrode when the sensed ultrasonic vibrations comprise a frequency within a pre-defined frequency range;
the assembly further comprising a frequency mixer for heterodyning the ultrasonic vibrations sensed by the sensing arrangement with a characteristic frequency.

2. A surgical assembly according to claim 1, wherein the pre-defined frequency range comprises a frequency associated with the ultrasonic vibrations delivered by an ultrasonic surgical device.

3. A surgical assembly according to claim 1 wherein the sensing arrangement comprises a transducer for capturing ultrasonic vibrations and a passband filter for removing ultrasonic vibrations which comprise a frequency outside the pre-defined range.

4. A surgical assembly according to claim 3, wherein the sensing arrangement further comprises a threshold detector for detecting the ultrasonic vibrations admitted by the passband filter, within the pre-defined frequency range, which comprise an amplitude above a threshold level.

5. A surgical assembly according to claim 4, wherein the threshold detector is further arranged to detect the ultrasonic vibrations within the pre-defined frequency range which exist for a temporal duration greater than a threshold duration.

6. A surgical assembly according to claim 4, wherein the sensing arrangement is arranged to output the activation signal when the ultrasonic vibrations comprise a frequency within the pre-defined frequency range and when the ultrasonic vibrations comprise an amplitude above the threshold level.

7. A surgical assembly according to claim 5, wherein the sensing arrangement is arranged to output the activation signal when the ultrasonic vibrations comprise a frequency within the pre-defined frequency range and when the ultrasonic vibrations comprise an amplitude above the threshold level and when the ultrasonic vibrations exist for a temporal duration greater than the threshold duration.

8. A surgical assembly according to claim 1, wherein the predefined frequency range comprises the range between 20 kHz and 70 kHz.

9. A surgical assembly according to claim 1, wherein the predefined frequency range comprises the range between 30 kHz and 60 kHz.

10. (canceled)

11. A surgical assembly according to claim 1, wherein the characteristic frequency comprises a frequency of ultrasonic vibrations generated by a known ultrasonic surgical device, so that the ultrasonic surgical device can be identified.

12. A surgical assembly according to claim 11, wherein the characteristic frequency comprises at least one frequency from the group comprising 36 kHz, 47 kHz and 55 kHz.

13. A surgical system comprising a surgical assembly according to claim 1, an electrical generator electrically coupled with the ion-generating electrode, for delivering the electrical signal to the ion-generating electrode, and an ultrasonic energy generator, communicatively coupled with an ultrasonic surgical device for delivering ultrasonic signal to the ultrasonic surgical device.
